(19) United States
(12) Patent Application Publication
(10) Pub. No.: US 2005/0246004 A1
(43) Pub. Date: Nov. 3, 2005

(54) Combination lead for electrical stimulation and sensing

(75) Inventors: Tracy L. Cameron, Toronto (CA); Terry D. Daglow, Allen, TX (US); Damon R. Black, Dallas, TX (US)

Correspondence Address: BAKER BOTTS L.L.P., 2001 ROSS AVENUE, 6TH FLOOR, DALLAS, TX, 75201-2980, US

(73) Assignee: Advanced Neuromodulation Systems, Inc.

(21) Appl. No.: 11/116,557

(22) Filed: Apr. 28, 2005

Related U.S. Application Data
(60) Provisional application No. 60/566,373, filed on Apr. 28, 2004.

(51) Int. Cl. A61N 1/05

(52) U.S. Cl. 607/116

(57) ABSTRACT

In one embodiment, an electrical stimulation lead is adapted for implantation in a person's body to provide therapeutic electrical stimulation of target nerve tissue within the person's body. The stimulation lead includes one or more stimulating electrodes and one or more sensing electrodes integrated into the stimulation lead and adapted for implantation in the person's body with the stimulation lead. The stimulating electrodes are operable to provide electrical stimulation to target nerve tissue within the person's body. The sensing electrodes are operable to detect electrical signals produced by nerve cells within the person's body to facilitate precise positioning of the stimulating electrodes proximate the target nerve tissue during implantation of the stimulation lead.

### RELATED APPLICATIONS

This application claims the benefit under 35 U.S.C. § 119(e) of U.S. Provisional Application Ser. No. 60/566,373, filed Apr. 28, 2004.

### TECHNICAL FIELD

This invention relates generally to electrical stimulation leads for medical purposes and in particular to combination leads for electrical stimulation and sensing.

### BACKGROUND

Electrical energy may be applied at various locations in a person's central nervous system, including locations within the brain, brain stem, spinal cord and peripheral nerves, for example, to treat a variety of clinical conditions such as movement disorders, chronic pain, or other conditions such as tinnitus. For example, deep brain stimulation may be used to reduce or prevent tremor from a movement disorder such as Parkinson's Disease. As another example, electrical energy may be applied to the spinal cord or a peripheral nerve to cause a subjective sensation of numbness or tingling in an affected region of the body, known as “paresthesia.” A variety of other clinical conditions may also be treated with deep brain stimulation, such as essential tremor, tremor from multiple sclerosis or brain injury, or dystonia or other movement disorders.

The electrical energy is delivered through stimulating electrodes positioned proximate the nerve tissue to be stimulated. The stimulating electrodes may be carried by either of two primary vehicles: a percutaneous lead and a laminotomy or “paddle” lead. Percutaneous leads typically have a number of equally-spaced circumferential stimulating electrodes. Percutaneous leads are positioned using a needle that is passed through the skin and, for example, into the brain such that the stimulating electrodes are proximate brain tissue targeted for stimulation. Percutaneous leads deliver energy radially in all directions because of the circumferential nature of the stimulating electrodes. Paddle leads have a paddle-like configuration and typically have multiple circumferential electrodes arranged in one or more columns. Paddle leads provide more focused energy delivery than percutaneous leads because circumferential electrodes may be present on only one surface of the lead. Paddle leads may be desirable in certain situations because they provide more direct stimulation to specific nerve tissue and require less energy to produce a desired effect.

In order to properly position the lead such that the stimulating electrodes are positioned proximate the nerve tissue targeted for stimulation, sensing electrodes may be used to determine the location of the lead within the person's body. For example, a lead may be hollow and have an opening in the tip to allow a stiffener having one or more sensing microelectrodes located at its tip to be inserted and removed through the opening in the tip of the lead. Thus, the lead may be inserted into the person's body through a cannula and positioned in a trial position. The stiffener is then inserted through the hollow lead such that the tip of the stiffener, at which the sensing microelectrodes are located, extends through the opening in the tip of the lead. The sensing microelectrodes are then used to detect the cell activity of cells proximate the sensing microelectrodes. If the user determines from the detected cell activity that the lead is not properly positioned, the stiffener is withdrawn such that the tip of the stiffener does not extends through the opening in the lead and the lead is moved to a new location within the person's body. The stiffener is then reinserted through the opening in the tip of the lead and the surrounding cell activity is detected. Once the user determines from the detected cell activity that the lead is properly positioned, the stiffener is removed from the person's body and the lead is secured in place.

### SUMMARY OF THE INVENTION

The present invention provides combination leads for electrical stimulation and sensing.

In one embodiment, an electrical stimulation lead is adapted for implantation in a person's body to provide therapeutic electrical stimulation of target nerve tissue within the person's body. The stimulation lead includes one or more stimulating electrodes and one or more sensing electrodes integrated into the stimulation lead and adapted for implantation in the person's body with the stimulation lead. The stimulating electrodes are operable to provide electrical stimulation to target nerve tissue within the person's body. The sensing electrodes are operable to detect electrical signals produced by nerve cells within the person's body to facilitate precise positioning of the stimulating electrodes proximate the target nerve tissue during implantation of the stimulation lead.

In another embodiment, a stimulation system for providing therapeutic electrical stimulation to target nerve tissue in a person's body is provided. The stimulation system includes a stimulation source operable to generate and transmit electrical stimulation pulses and an electrical stimulation lead adapted for implantation into a person's body for electrical stimulation of target nerve tissue within the person's body. The stimulation lead includes one or more sensing electrodes and one or more stimulating electrodes integrated into the stimulation lead and adapted for implantation in the person's body with the stimulation lead. The one or more stimulating electrodes are operable to deliver the electrical stimulation pulses to target nerve tissue within the person's body to provide therapeutic relief to a region of the person's body corresponding to the target nerve tissue. The one or more sensing electrodes are operable to detect electrical signals produced by nerve cells within the person's body to facilitate precise positioning of the one or more stimulating electrodes proximate the target nerve tissue during implantation of the stimulation lead.

In another embodiment, a method is provided for locating an electrical stimulation lead proximate target nerve tissue within a person's body for providing therapeutic stimulation to the target nerve tissue. An electrical stimulation lead including one or more sensing electrodes and one or more stimulating electrodes integrated into the stimulation lead is inserted into the person's body. Electrical signals are generated by nerve cells proximate the sensing electrodes using the one or more sensing electrodes, and signals reflecting the detected electrical signals is transmitted from the one or more sensing electrodes. The precise location of the one or more sensing electrodes within the body is determined based at least in part on the signals reflecting the detected electrical signals. The stimulation lead is positioned until the one or more stimulating electrodes are located proximate the target nerve tissue based on the determined precise location of the one or more sensing electrodes. The stimulation lead is secured in position when the one or more stimulating electrodes are located proximate the target nerve tissue, and electrical stimulation pulses generated by a stimulation source are delivered to the target nerve tissue to provide therapeutic relief to a region of the person's body corresponding to the target nerve tissue.

In another embodiment, a method for providing therapeutic electrical stimulation within a person's body is provided. Electrical stimulation signals are generated and transmitted using a stimulation source implanted within the person's body. The electrical stimulation signals generated by the stimulation source are delivered to target nerve tissue within the person's body using one or more stimulating electrodes integrated into an electrical stimulation lead implanted in the person's body and coupled to the stimulation source. The electrical stimulation signals provide therapeutic relief to a region of the person's body corresponding to the target nerve tissue. Using one or more sensing electrodes integrated into the electrical stimulation lead implanted in the person's body, electrical signals generated by nerve cells proximate the one or more sensing electrodes are detected. Signals reflecting the detected electrical signals is transmitted and one or more stimulation parameters of the electrical stimulation pulses generated and transmitted by the stimulation source are controlled based at least on the signals reflecting the detected electrical signals.

Particular embodiments of the present invention may provide one or more technical advantages. For example, in certain embodiments, stimulating electrodes and sensing electrodes are integrated into a single implantable stimulation lead. Thus, a single stimulation lead may be used for both detection of nerve cell activity and stimulation of target nerve tissue for therapeutic purposes. The detection of nerve cell activity may be used for determining the precise location of the stimulation lead within the person's body during implantation and positioning of the stimulation lead. Once the stimulation lead is precisely positioned within the body such that the stimulating electrodes are located proximate the target nerve tissue, the stimulation lead may be secured in position with the sensing electrodes remaining in the body. Thus, by including sensing electrodes integrated into the stimulation lead, the stimulation lead may be more efficiently and more effectively located proximate the target nerve tissue as compared with prior stimulation leads and methods for positioning such stimulation leads. In addition, in certain embodiments, the sensing electrodes may be used for continued sensing of nerve cell activity during operation of the stimulation system to provide feedback that may be used as input for controlling one or more stimulation parameters of the electrical stimulation pulses delivered to the target nerve tissue by the stimulating electrodes.

Certain embodiments may provide all, some, or none of these advantages. Certain embodiments may provide one or more other advantages, one or more of which may be apparent to those skilled in the art from the figures, descriptions, and claims included herein.

### BRIEF DESCRIPTION OF THE DRAWINGS

To provide a more complete understanding of the present invention and the features and advantages thereof, reference is made to the following description taken in conjunction with the accompanying drawings, in which:

FIGS. 1A-1B illustrate example electrical stimulation systems for providing therapeutic electrical stimulation of a target nerve tissue in a person's body;

FIG. 2 illustrates an example monitoring system for processing signals received from sensing electrodes that reflect the activity of nerve cells in a person's body;

FIGS. 3A-3B illustrate side and end views, respectively, of the stimulating portion of an example percutaneous stimulation lead having at least one stimulating electrode and a plurality of sensing electrodes;

FIGS. 4A-4B illustrate side and cross-sectional views, respectively, of the stimulating portion of an example percutaneous stimulation lead having at least two stimulating electrodes and a plurality of sensing electrodes;

FIGS. 5A-5I illustrate various other example stimulation leads, each having at least one stimulating electrode and at least one sensing electrode;

FIGS. 6A-6B illustrate an example of a person undergoing placement of a stimulation lead for electrical stimulation of target nerve tissue within a person's brain;

FIG. 7 illustrates an example method for implanting a stimulation lead in a person's brain such that stimulating electrodes are located proximate target nerve tissue in the brain;

FIG. 8 illustrates an example method of using sensing electrodes for continued detection of nerve cell activity during operation of the stimulation system;

FIG. 9 illustrates an example stimulation set;

FIG. 10 illustrates a number of example stimulation programs, each of which includes a number of stimulation sets; and

FIG. 11 illustrates example execution of a sequence of stimulation sets within an example stimulation program.

### DESCRIPTION OF EXAMPLE EMBODIMENTS

According to the present invention, an implantable electrical stimulation lead for providing electrical stimulation to target nerve tissue in a person's body includes both stimulating electrodes and sensing electrodes integrated into, and adapted for implantation in the person's body along with, the stimulation lead. Thus, both the stimulating electrodes and sensing electrodes provided with the stimulation lead remain within the person's body after the stimulation lead has been implanted.

The sensing electrodes detect electrical signals produced by nerve cells in the person's body, allowing signals reflecting the detected signals to be used to determine the precise location of the stimulation lead within the person's body. Thus, the sensing electrodes may be used for precise positioning of the stimulation lead within the person's body such that the stimulating electrodes are located proximate the target nerve tissue. Once the stimulation lead is properly positioned and secured, the stimulating electrodes deliver electrical stimulation pulses generated by a stimulation source to the target nerve tissue to treat any of a variety of clinical conditions experienced by the person, such as movement disorders, chronic pain, or other conditions such as tinnitus. In certain embodiments, the sensing electrodes continue to detect electrical signals produced by nerve cells in the person's body and to transmit signals reflecting the detected electrical signals. One or more stimulation parameters of the electrical stimulation pulses generated by the stimulation source and applied to the target nerve tissue by the stimulating electrodes may be adjusted based at least in part on the signals reflecting the detected electrical signals.

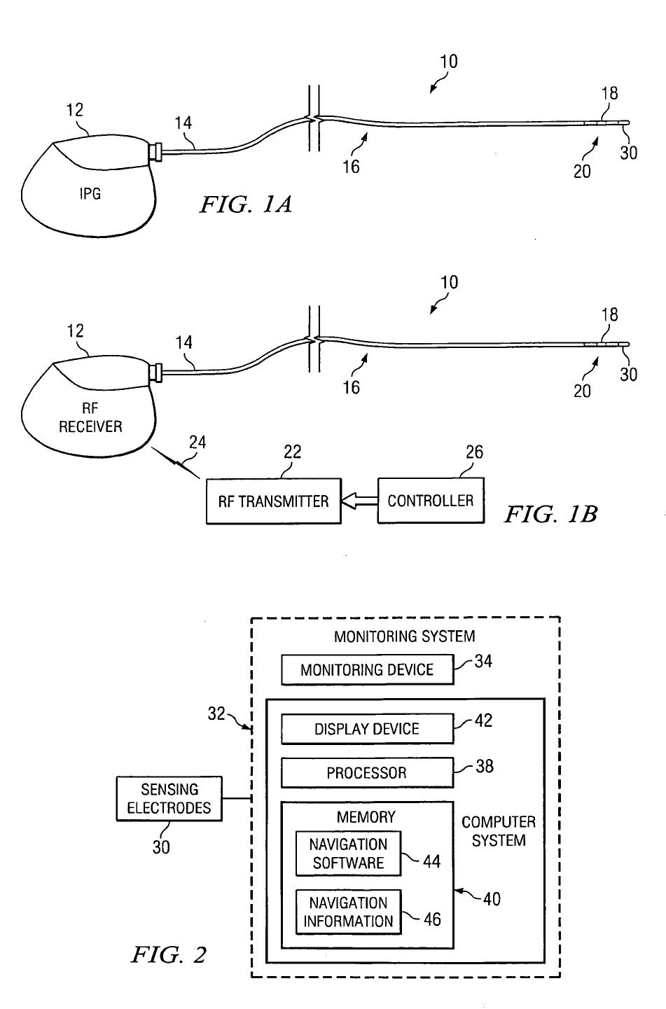

FIGS. 1A-1B illustrate example electrical stimulation systems 10 for providing electrical stimulation of target nerve tissue in a person's body to treat a variety of clinical conditions experienced by the person, such as movement disorders, chronic pain, or other conditions such as tinnitus. In certain embodiments, the target nerve tissue includes one or more groups of cells within the person's central nervous system, such as groups of nerve cells within the brain, the brain stem, the spinal cord or a peripheral nerve, for example.

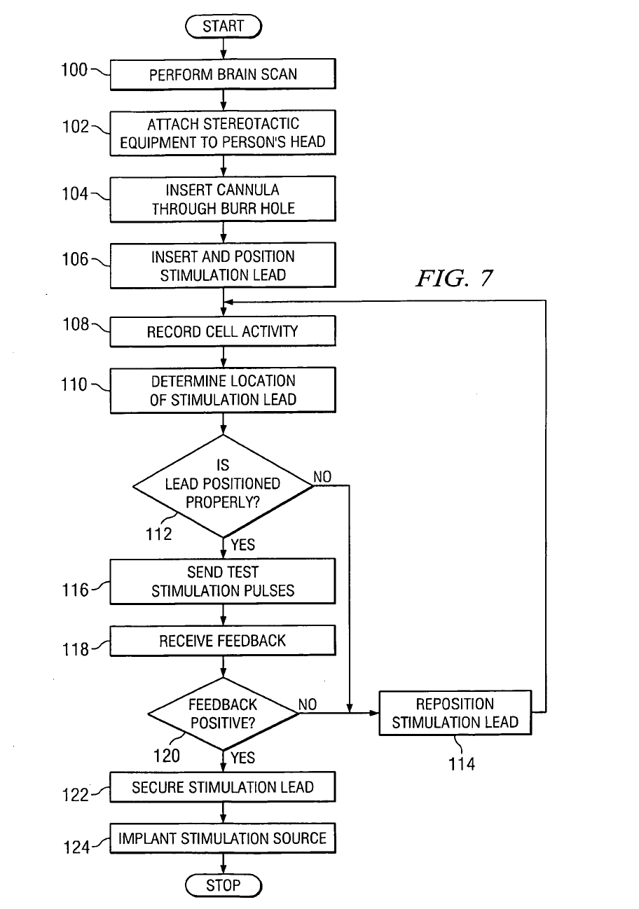

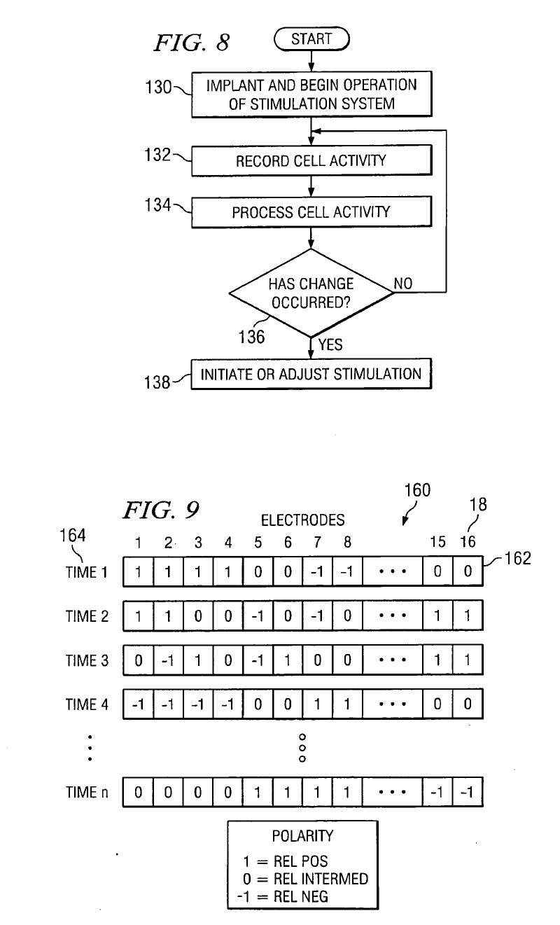

Stimulation system 10 includes an implantable electrical stimulation source 12 and an implantable electrical stimulation lead 14 for applying electrical stimulation pulses to the target nerve tissue. In operation, both of these primary components are implanted in the person's body, as discussed below with reference to FIGS. 7-8. Stimulation source 12 is coupled to a connecting portion 16 of electrical stimulation lead 14. Stimulation source 12 controls the electrical stimulation pulses transmitted to one or more stimulating electrodes 18 integrated into a stimulating portion 20 of electrical stimulation lead 14, located proximate the target nerve tissue, according to suitable stimulation parameters (e.g., duration, intensity, frequency, etc.). Stimulating electrodes 18 may be microelectrodes, semi-microelectrodes, or macroelectrodes according to particular needs. Stimulating electrodes 18 are integrated into, and adapted for implantation in the person's body along with, stimulation lead 14. A doctor, the patient, or another user of stimulation source 12 may directly or indirectly input stimulation parameters for controlling the nature of the electrical stimulation pulses provided.

In certain embodiments, stimulation source 12 may produce electrical stimulation pulses according to one or more stimulation programs, each including a number of stimulation sets. Each stimulation set may specify a number of stimulation parameters for that stimulation set. Stimulation parameters may include, for example, an amplitude (or intensity), a frequency, phase information, and a pulse width for each of a series of stimulation pulses that stimulating electrodes 18 are to deliver to the target nerve tissue during a particular time interval, along with a polarity for each stimulating electrode 18 during each stimulation pulse. Stimulation parameters may also include a pulse shape, for example, biphasic cathode first, biphasic anode first, or any other suitable pulse shape. Various stimulation programs, stimulation sets and stimulation parameters associated with the electrical stimulation pulses produced by stimulation source 12 are discussed in greater detail below with reference to FIGS. 9-11.

In one embodiment, as shown in FIG. 1A, stimulation source 12 includes an implantable pulse generator (IPG). An example IPG may be one manufactured by Advanced Neuromodulation Systems, Inc., such as the Genesis® System, part numbers 3604, 3608, 3609, and 3644. In another embodiment, as shown in FIG. 1B, stimulation source 12 includes an implantable wireless receiver. An example wireless receiver may be one manufactured by Advanced Neuromodulation Systems, Inc., such as the Renew® System, part numbers 3408 and 3416. The wireless receiver is capable of receiving wireless signals from a wireless transmitter 22 located external to the person's body. The wireless signals are represented in FIG. 1B by wireless link symbol 24. A doctor, the patient, or another user of stimulation source 12 may use a controller 26 located external to the person's body to provide control signals for operation of stimulation source 12. Controller 26 provides the control signals to wireless transmitter 22, wireless transmitter 22 transmits the control signals and power to the wireless receiver of stimulation source 12, and stimulation source 12 uses the control signals to vary the stimulation parameters of electrical stimulation pulses generated by stimulation source 12 and delivered by stimulating electrodes 18 to the target nerve tissue. An example wireless transmitter 22 may be one manufactured by Advanced Neuromodulation Systems, Inc., such as the Renew® System, part numbers 3508 and 3516.

In addition to stimulating electrodes 18, one or more sensing electrodes 30 are also integrated into, and adapted for implantation in the person's body along with, stimulation lead 14. In certain embodiments, sensing electrodes 30 are located at or near stimulating portion 20 of electrical stimulation lead 14. Sensing electrodes 30 are generally operable to detect electrical signals produced by nerve cells in a person's body, such as when a nerve cell fires an electrical impulse, for example, and to transmit signals reflecting the detected electrical signals. For example, the signals transmitted by sensing electrodes 30 that reflect the detected electrical signals may indicate one or more parameters, the amplitude (or intensity) for example, of the detected electrical signals. As another example, the signals transmitted by sensing electrodes 30 may include the actual detected signals themselves. However, it should be understood that the signals transmitted by sensing electrodes 30 may include any other suitable signals that reflect or are otherwise associated with electrical signals produced by nerve cells proximate sensing electrodes 30 and detected by sensing electrodes 30.

Like stimulating electrodes 18, sensing electrodes 30 may be microelectrodes, semi-microelectrodes, or macroelectrodes according to particular needs. In certain embodiments, stimulating electrodes 18 are macroelectrodes and sensing electrodes 30 are microelectrodes or semi-microelectrodes. In addition, it should be understood that in certain implementations of stimulation system 10, the target nerve tissue stimulated by stimulating electrodes 18 may include one or more nerve cells being monitored by sensing electrodes 30.

In operation, sensing electrodes 30 may provide various functionality. For example, sensing electrodes 30 may be used to detect nerve cell activity and transmit signals reflecting such nerve cell activity which may be used to determine the precise location of stimulation lead 14, which may be useful for precisely positioning stimulation lead 14 within the person's body, as discussed in greater detail below with reference to FIG. 7. Detecting information regarding nerve cell activity may include detecting electrical signals produced by nerve cells proximate sensing electrodes 30. As another example, sensing electrodes 30 may be used for continued detection of electrical signals produced by nerve cells after stimulation lead 14 has been implanted to provide feedback to stimulation system 10, which may be used to determine whether to initiate an event or to adjust one or more stimulation parameters of the stimulation pulses generated by stimulation source 12, as discussed in greater detail below with reference to FIG. 8.

FIG. 2 illustrates an example monitoring system 32 for processing signals received from sensing electrodes 30 that reflect the activity of nerve cells in a person's body according to certain embodiments of the present invention. Monitoring system 32 may include a monitoring device 34 and/or a computer system 36, each generally operable to process signals reflecting electrical signals detected by sensing electrodes 30. Sensing electrodes 30 are integrated into stimulation lead 14, as discussed above.

Monitoring device 34 may comprise an oscilloscope or other device suitable to receive electrical signals from sensing electrodes 30 and to generate an output on a display corresponding to the received signals reflecting the electrical signals. Monitoring device 34 may be permanently or temporarily connected to stimulation lead 14. For example, monitoring device 34 may be integrated with stimulation source 12 or may be temporarily connected to connecting portion 16 of stimulation lead 14 during the process of positioning stimulation lead 14 but removed from connecting portion 16 and replaced with stimulation source 12 once stimulation lead 14 is properly positioned within the body.

As discussed in greater detail below with reference to FIG. 7, monitoring device 34 may be used during the implantation and positioning of stimulation lead 14. For example, during the implantation and positioning of stimulation lead 14, sensing electrodes 30 may detect electrical signals produced by nerve cells proximate sensing electrodes 30 and transmit signals reflecting the detected electrical signals to monitoring device 34. Monitoring device 34 may receive and process these transmitted signals and generate an output, such as a display for example, that may be used by a doctor or other user to determine the precise location of sensing electrodes 30 within the person's body. The determined precise location of sensing electrodes 30 may indicate the precise positioning of stimulating electrodes 18 based on knowledge of the spatial relationship between sensing electrodes 30 and stimulating electrodes 18. Alternatively, in certain embodiments, monitoring device 34 may process the received signals reflecting the detected electrical signals and automatically determine the precise location of sensing electrodes 30 and/or stimulating electrodes 18 within the person's body, such as by comparing the signals reflecting the detected electrical signals with navigation information regarding the location of nerve tissue in the person's body stored in monitoring device 34. Thus, sensing electrodes 30 and monitoring device 34 may be used for precise positioning of stimulation lead 14 within the person's body such that the stimulating electrodes 18 are located proximate the target nerve tissue.

Instead or in addition, monitoring device 34 may be used for continued monitoring of nerve cell activity during the operation of stimulation system 10, as discussed in greater detail below with reference to FIG. 8. For example, once stimulation lead 14 is implanted and secured in position, sensing electrodes 30 integrated into stimulation lead 14 may detect electrical signals produced by nerve cells proximate sensing electrodes 30 and transmit signals reflecting the detected electrical signals to monitoring device 34. Monitoring device 34 may receive and process the transmitted signals and determine whether to control the electrical stimulation pulses generated by stimulation source 12 and delivered to the target nerve tissue by stimulating electrodes 18. Such control may include causing stimulation source 12 to initiate a stimulation event, such as to begin sending electrical stimulation pulses or to adjust one or more stimulation parameters, such as the duration, amplitude (or intensity), or frequency for example, of stimulation pulses generated by stimulation source 12. In certain embodiments, monitoring device 34 may be integrated with stimulation source 12 and adapted for implantation within the person's body.

In certain embodiments, monitoring device 34 may be operable to relay signals received from sensing electrodes 30 to computer system 36 to process such signals. For example, in embodiments in which monitoring device 34 is integrated into stimulation source 12, computer system 36 may be connected to monitoring device 34 via an output port located on stimulation source 12. In other embodiments, computer system 36 may be temporarily connected to an output port located on stimulation source 12 and may receive signals directly from sensing electrodes 30 through the output port.

Computer system 36 is generally operable to process signals received from sensing electrodes 30 or from monitoring device 34 to facilitate the precise positioning of stimulation lead 14 within the person's body such that stimulating electrodes 18 are positioned proximate the target nerve tissue. Computer system 36 may include a processor 38, memory 40, and a display device 42. Computer system 36 may include any suitable components operable to accept input, process the input according to predefined rules, and produce output to display device 42. Display device 42 may be any suitable device for displaying information to a user at any suitable location local to or remote from other components of computer system 36.

Memory 40 may include any suitable data storage arrangement and may be used to store navigation software 44 and navigation information 46. Navigation information 46 may include at least information regarding the location of various nerve tissue within the person's body and/or nerve cell activity associated with such nerve tissue. In certain embodiments, navigation information 46 may include information about the body of a particular person that is obtained from a magnetic resonance imaging (MRI) scan, a functional MRI (fMRI) scan, or other type of scan of the person's body. Navigation software 44 may be executed by processor 38 and may compare signals received from sensing electrodes 30 or from monitoring device 34 with navigation information 46 to determine the precise location of sensing electrodes 30 within the person's body. Navigation software 44 may generate information for display on display device 42 that indicates the precise location of sensing electrodes 30 within the person's body. Thus, a doctor or other user may view display device 42 while implanting stimulation lead 14 within the person's body to determine the precise location of sensing electrodes 30, which may further indicate the precise positioning of stimulating electrodes 18 based on knowledge of the spatial relationship between sensing electrodes 30 and stimulating electrodes 18.

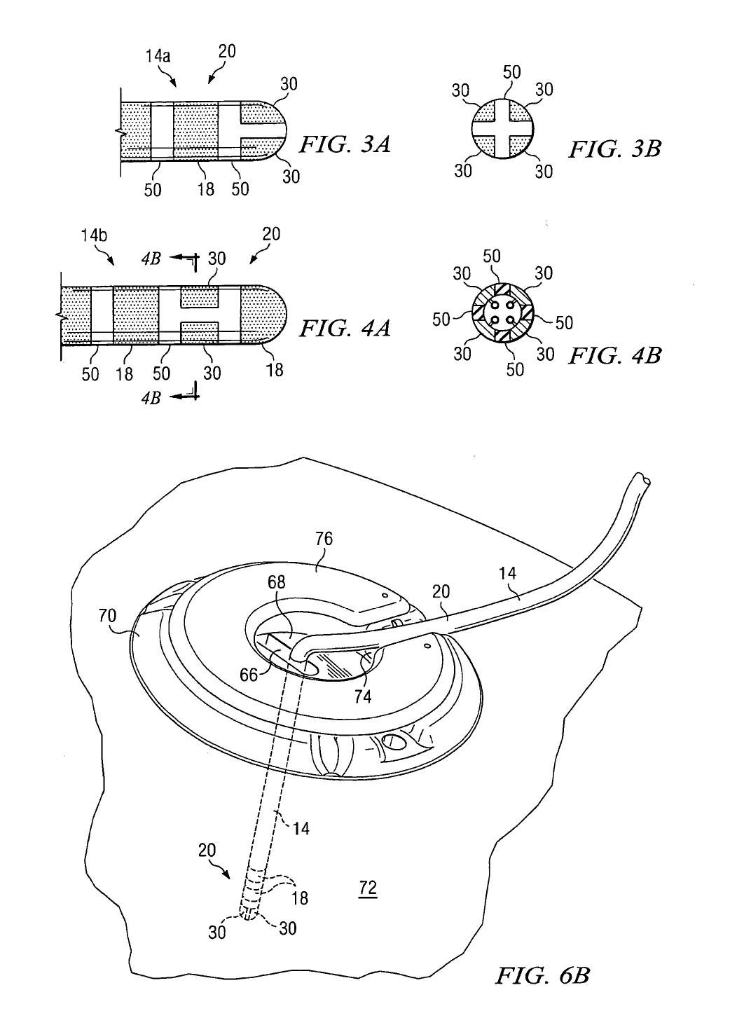

FIGS. 3A-3B illustrate side and end views, respectively, of the stimulating portion 20 of an example percutaneous stimulation lead 14a having at least one stimulating electrode 18 and a plurality of sensing electrodes 30. One or more circumferential macroelectrode stimulating electrodes 18 are located in series along stimulating portion 20 of stimulation lead 14a, and four semi-microelectrode sensing electrodes 30 are spaced apart form one another around the tip of stimulation lead 14a. Circumferential stimulating electrodes 18 emit electrical stimulation energy generally radially in all directions. Similarly, sensing electrodes 30 are located around the circumference of the tip of stimulation lead 14a to detect electrical signals from all directions around the tip of stimulation lead 14a. In other embodiments, more or less than four semi-microelectrode sensing electrodes 30 are located around the tip of stimulation lead 14a. Stimulating electrodes 18 and sensing electrodes 30 are electrically insulated from each other by a dielectric material 50. Further, each stimulating electrode 18 and sensing electrode 30 is connected to stimulation source 12 by a separate insulated wire to prevent shorting between the various electrodes.

FIGS. 4A-4B illustrate the stimulating portion 20 of another example percutaneous stimulation lead 14b having at least two stimulating electrode 18 and a plurality of sensing electrodes 30. In particular, FIG. 4A illustrates a side view of stimulation lead 14b, while FIG. 4B illustrates a cross section of stimulation lead 14b taken along line 4B-4B shown in FIG. 4A. As shown in FIGS. 4A and 4B, four semi-microelectrode sensing electrodes 30 are spaced apart form one another around the circumference of stimulation lead 14b between a pair of circumferential macroelectrode stimulating electrodes 18. As discussed above, circumferential stimulating electrodes 18 emit electrical stimulation energy generally radially in all directions, and sensing electrodes 30 are located around the circumference of stimulation lead 14a to detect electrical signals from all directions around stimulation lead 14a. In other embodiments, more or less than four semi-microelectrode sensing electrodes 30 are located around the circumference of stimulation lead 14b. Stimulating electrodes 18 and sensing electrodes 30 are electrically insulated from each other by a dielectric material 50. Further, each stimulating electrode 18 and sensing electrode 30 is connected to stimulation source 12 by a separate insulated wire to prevent shorting between the various electrodes. For example, as shown in FIG. 4B, each sensing electrode 30 may be connected to a separate insulated wire 52 by a laser weld.

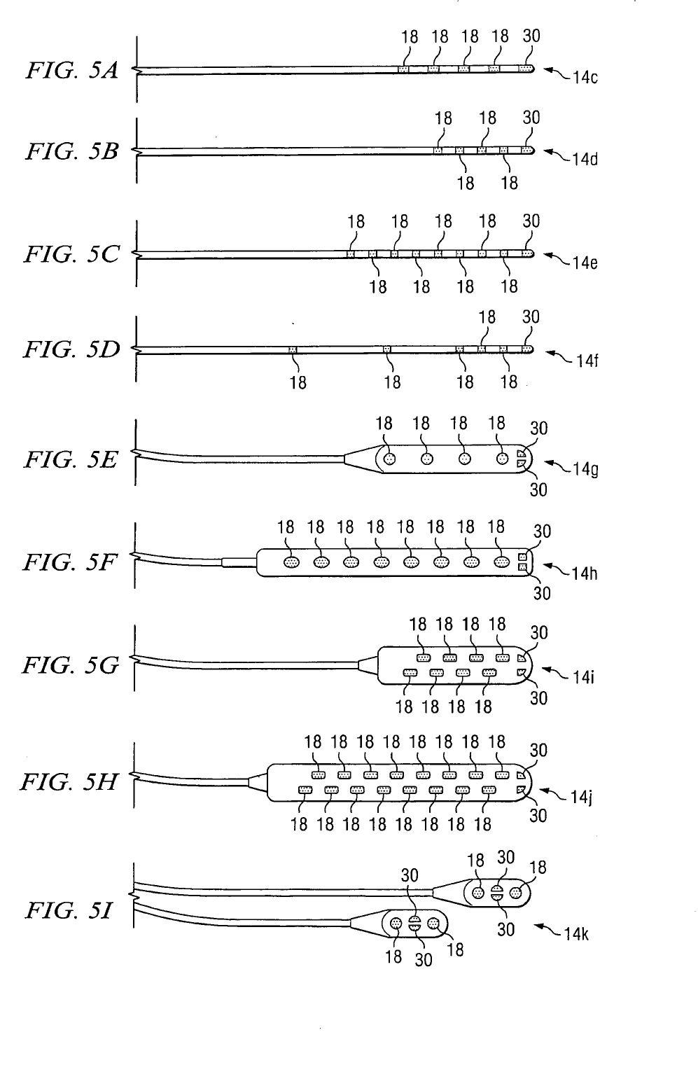

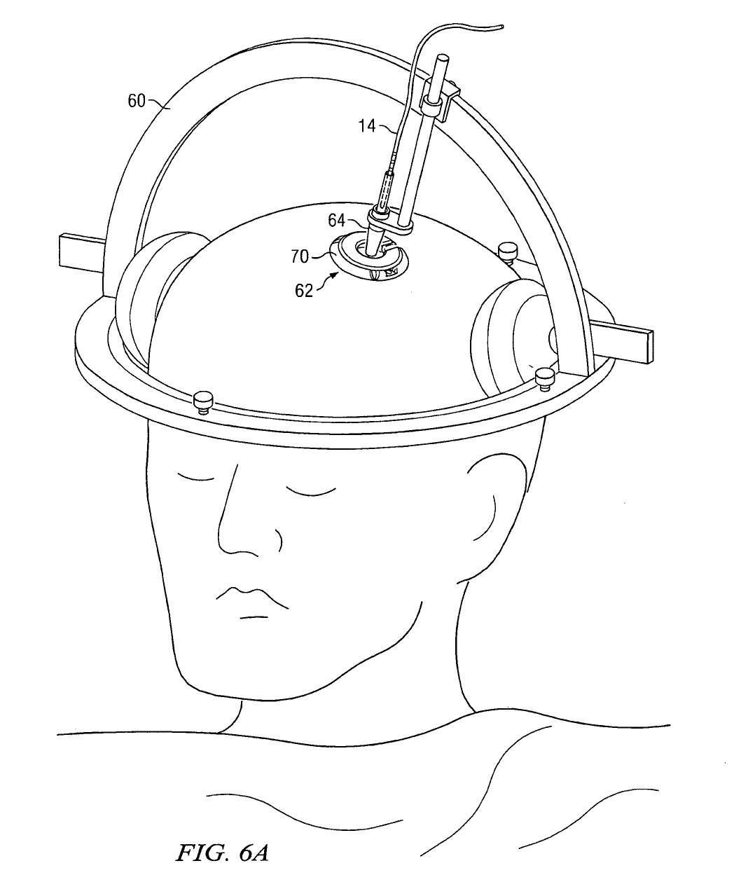

FIGS. 5A-5I illustrate various other example stimulation leads 14, each having at least one stimulating electrode 18 and at least one sensing electrode 30. As discussed above, stimulating electrodes 18 on each stimulation lead 14 are adapted to be positioned near the target nerve tissue and used to deliver electrical stimulation pulses received from stimulation source 12 to the target nerve tissue. Sensing electrodes 30 on each stimulation lead 14 are adapted to detect electrical signals produced by nerve cells proximate sensing electrodes 30 and to transmit signals reflecting the detected electrical signals, which may be used for precisely positioning stimulation lead 14 proximate the target nerve tissue and/or for continued monitoring of nerve cell activity during the operation of stimulation system 10.

Example stimulation leads 14c-f (as well as stimulation leads 14a and 14b shown in FIGS. 3 and 4) are percutaneous stimulation leads that include one or more circumferential stimulating electrodes 18 spaced apart from one another along the stimulating portion 20 of stimulation lead 14, as well as a plurality of sensing electrodes 30 spaced apart from one another around the circumference of stimulation lead 14 at its tip. As discussed above, circumferential stimulating electrodes 18 emit electrical stimulation energy generally radially in all directions, and sensing electrodes 30 are located around the circumference of stimulation lead 14a to detect electrical signals generally radially in all directions.

Example stimulation leads 14g-k are paddle stimulation leads that include one or more directional stimulating electrodes 18 and one or more directional sensing electrodes 30 spaced apart from one another along one surface of stimulating portion 20 of stimulation lead 14. Directional stimulating electrodes 18 emit electrical stimulation energy, and sensing electrodes 30 detect electrical signals produced by nerve cells, in a direction generally perpendicular to the surface of stimulation lead 14 on which they are located. Although various types of stimulation leads 14 are shown as examples, the present invention contemplates stimulation system 10 including any suitable type of stimulation lead 14 in any suitable number. In addition, two or more stimulation leads 14 may be used in combination.

FIGS. 6A-6B illustrate an example of a person undergoing insertion and positioning of an electrical stimulation lead 14 for stimulation of target nerve tissue within the person's brain using stereotactic equipment 60 to guide the insertion and positioning of stimulation lead 14 and an apparatus 62 to secure stimulation lead 14 in position in the person's brain. As can be appreciated from FIG. 6A, electrical stimulation lead 14 is typically coupled to stereotactic equipment 60 during the lead placement for increased stability and housed within an insertion cannula 64 for insertion into the brain. Stimulation lead 14 is inserted through a burr hole in the skull and navigated to its target position using signals received from sensing electrodes 30 that reflect the nerve cell activity of nerve cells proximate sensing electrodes 30, as discussed in greater detail below with reference to FIG. 7.

FIG. 6B shows a close-up view of stimulating portion 20 of stimulation lead 14, with both stimulating electrodes 18 and sensing electrodes 30, after insertion through a slot 66 in a disc 68 located within a ring 70 and subsequent removal of cannula 64. Stimulating electrodes 18 and sensing electrodes 30 are located proximate target nerve tissue within the brain, indicated generally at 72, such that stimulating electrodes 18 may deliver stimulation pulses to target nerve tissue 72. The connecting portion 20 of electrical stimulation lead 14 is positioned in a transverse channel 74 of ring 70 to lay substantially flat on the skull. A removable cap 76 is coupled to ring 70 to secure disc 68 in position and to additionally help to prevent both leakage from the burr hole and entry of contaminants into the burr hole where appropriate.

FIG. 7 illustrates an example method for implanting stimulation lead 14 in a person's brain such that stimulating electrodes 18 are located proximate target nerve tissue 72. The method discussed below represents only an example implementation of stimulation lead 14; in other implementations, stimulation lead 14 may be implanted and positioned proximate other nerve tissue in a person's body, such as nerve tissue in the brain stem, the spinal cord or a peripheral nerve, for example.

At step 100, an MRI or fMRI scan of the person's brain is performed and the results of the MRI or fMRI are downloaded into a neuronavigation system, which may be associated with computer system 36 discussed above with reference to FIG. 2. At step 102, stereotactic equipment 60 may be attached to the person's head to guide the insertion and positioning of stimulation lead 14, and apparatus 62 may be positioned at a burr hole in the person's skull to secure stimulation lead 14 in position in the person's brain, as shown in FIGS. 6A-6B. At step 104, cannula 64 for electrical stimulation lead 14 may be inserted through the burr hole in the person's skull into the brain. However, a cannula is not typically used where stimulation lead 14 is a paddle lead. Cannula 64 and electrical stimulation lead 14 may be inserted together or stimulation lead 14 may be inserted through cannula 64 after cannula 64 has been inserted. Guided by the navigation system that includes the MRI or fMRI data obtained at step 100, stimulation lead 14 is inserted through cannula 64 and positioned within the brain at step 106.

At step 108, sensing electrodes 30 integrated into stimulation lead 14 detect electrical signals produced by nerve cells proximate sensing electrodes 30 and communicate signals reflecting the detected electrical signals to computer system 36 for processing. The signals reflecting the detected electrical signals is processed by monitoring device 34 and/or computer system 36 to determine the location of sensing electrodes 30 within the brain at step 110. This processing may involve a comparison of the signals reflecting the detected electrical signals with navigation information 46 regarding the location of nerve tissue and/or nerve cell activity for such nerve tissue in different regions of the person's brain, for example. The results of the processing may be displayed to a doctor or other user by display device 42 of computer system 36.

At step 112, an initial determination of whether stimulation lead 14 is located such that stimulating electrodes 18 are positioned proximate target nerve tissue 72 is made, either automatically by computer system 36 or by the doctor or other user viewing the results of the processing performed at step 110 on display device 42. If the initial determination at step 112 indicates that the stimulation lead 14 is not located such that stimulating electrodes 18 are positioned proximate target nerve tissue 72, the user may reposition stimulation lead 14 within the brain at step 114 and return to step 108 to record the nerve cell activity at this new location of stimulation lead 14.

Alternatively, if the initial determination at step 112 indicates that the stimulation lead 14 is located such that stimulating electrodes 18 are positioned proximate target nerve tissue 72, a test stimulation may be performed at step 116 by activating stimulation source 12, which generates and sends electrical pulses to the brain via stimulating electrodes 18. At step 118, the person indicates whether the person's affliction is suppressed by the test stimulation pulses. If the person's affliction is not suppressed at step 120, indicating that stimulating electrodes 18 are not positioned proximate target nerve tissue 72, the user may reposition stimulation lead 14 within the brain at step 114 and return to step 108 to record the nerve cell activity at this new location of stimulation lead 14. Alternatively, if the person's affliction is suppressed at step 120, indicating that stimulating electrodes 18 are positioned proximate target nerve tissue 72, stimulation lead 14 is uncoupled from stereotactic equipment 60 and secured in position, and cannula 64 and stereotactic equipment 60 are removed at step 122. Connecting portion 16 of electrical stimulation lead 14 is laid substantially flat along the person's skull. Where appropriate, any burr hole cover seated in the burr hole may be used to secure electrical stimulation lead 14 in position and possibly to help prevent leakage from the burr hole and entry of contaminants into the burr hole. Example burr hole covers that may be appropriate in certain embodiments are illustrated and described in copending U.S. application Ser. Nos. 11/010,108 and 11/010,136, both filed Dec. 10, 2004 and entitled “Electrical Stimulation System and Associated Apparatus for Securing an Electrical Stimulation Lead in Position in a Person's Brain.”

Once electrical stimulation lead 14 has been inserted and secured, stimulation source 12 is implanted at step 124. The implant site is typically a subcutaneous pocket formed to receive and house stimulation source 12. The implant site is usually positioned a distance away from the insertion site, such as near the chest area or buttocks or another place in the person's torso. Connecting portion 16 of stimulation lead 14 extends from the lead insertion site to the implant site at which stimulation source 12 is implanted. Once all appropriate components of stimulation system 10 are implanted, these components may be subject to mechanical forces and movement in response to movement of the person's body. A doctor, the patient, or another user of stimulation source 12 may directly or indirectly input signal parameters for controlling the nature of the electrical stimulation provided.

Although example steps are illustrated and described, the present invention contemplates two or more steps taking place substantially simultaneously or in a different order. In addition, the present invention contemplates using methods with additional steps, fewer steps, or different steps, so long as the steps remain appropriate for implanting an example stimulation system 10 into a person for electrical stimulation of target nerve tissue in the person's brain.

FIG. 8 illustrates an example method of using sensing electrodes 30 for continued recording of nerve cell activity after stimulation lead 14 and stimulation source 12 have been implanted in the person's body in an embodiment in which monitoring device 34 is integrated with stimulation source 12. The method discussed below represents only an example implementation of stimulation lead 14; in other implementations, stimulation lead 14 may be implanted and used for continued recording of nerve cell activity in other nerve tissue in a person's body, such as nerve tissue in the brain stem, the spinal cord or a peripheral nerve, for example.

At step 130, stimulation system 10 is implanted and begins operating to deliver electrical stimulation pulses to the target nerve tissue. At step 132, sensing electrodes 30 integrated into stimulation lead 14 detect electrical signals produced by nerve cells proximate sensing electrodes 30 and transmit signals reflecting the detected electrical signals to monitoring device 34. In certain implementations, the target nerve tissue stimulated by stimulating electrodes 18 at step 130 includes one or more of the nerve cells monitored by sensing electrodes 30 at step 132. At step 134, monitoring device 34 processes the signals reflecting the detected electrical signals and determines whether to control the electrical stimulation pulses generated by stimulation source 12 at step 136 based at least on the signals reflecting the detected electrical signals. For example, monitoring device 34 may determine whether to control the electrical stimulation pulses generated by stimulation source 12 based on whether the signals reflecting the detected electrical signals indicates a change in the activity of the nerve cells monitored by sensing electrodes 30.

Controlling the electrical stimulation pulses generated by stimulation source 12 may include causing stimulation source 12 to initiate a stimulation event, such as to begin sending electrical stimulation pulses, for example, or to adjust one or more stimulation parameters of ongoing stimulation pulses, such as the duration, amplitude (or intensity), or frequency for example, of stimulation pulses generated by stimulation source 12. Thus, for example, in an embodiment in which stimulation system 10 is used to provide paresthesia in an afflicted region of a person's body, if monitoring device 34 determines that a change in the activity of the nerve cells monitored by sensing electrodes 30 has occurred, monitoring device 34 may cause stimulation source 12 to adjust one or more stimulation parameters of the electrical stimulation pulses generated by stimulation source 12 to maintain a substantially constant level of paresthesia in the afflicted region of the person's body. Alternatively, if monitoring device 34 determines that the electrical stimulation pulses generated by stimulation source 12 need not be controlled based on the signals reflecting the detected electrical signals processed at step 134, the method may return to steps 132 and 134 to continue detecting subsequent electrical signals produced by the nerve cells proximate sensing electrodes 30, transmitting signals reflecting such detected electrical signals, and processing such transmitted signals to determine whether to control the electrical stimulation pulses generated by stimulation source 12.

Again, although example steps are illustrated and described, the present invention contemplates two or more steps taking place substantially simultaneously or in a different order. In addition, the present invention contemplates using methods with additional steps, fewer steps, or different steps, so long as the steps remain appropriate for using one or more sensing electrodes 30 for continued recording of nerve cell activity during the operation of stimulation system 10.

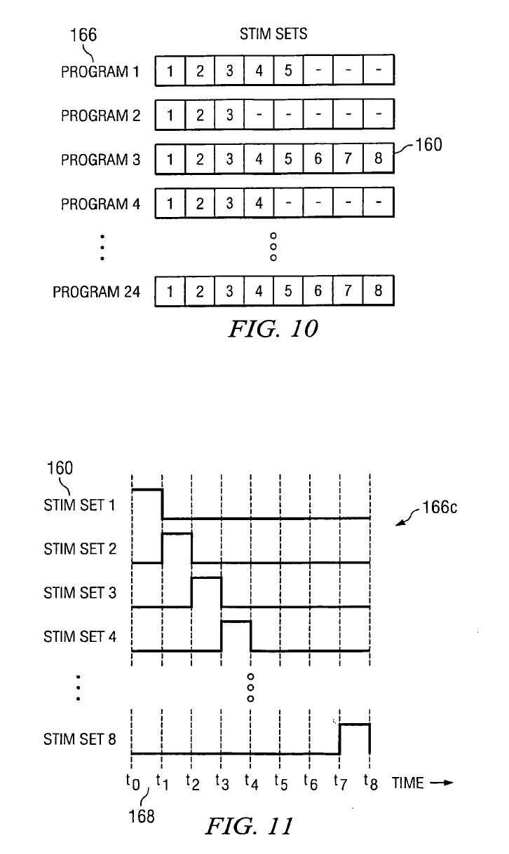

FIG. 9 illustrates an example stimulation set 160. One or more stimulation sets 160 may be provided, each stimulation set 160 specifying a number of stimulation parameters for the stimulation set 160. For example, as described more fully below with reference to FIGS. 10-11, multiple stimulation sets 160 may be executed in a suitable sequence according to a pre-programmed or randomized stimulation program. Example stimulation parameters for a stimulation set 160 may include an amplitude (or intensity), a frequency, phase information, and a pulse width for each of a series of stimulation pulses that stimulating electrodes 18 are to deliver to the target nerve tissue during a time interval during which stimulation set 160 is executed, along with a polarity 162 for each stimulating electrode 18 within each stimulation pulse. In general, in particular embodiments in which stimulation lead 14 includes two or more stimulating electrodes 18, electric fields are generated between adjacent stimulating electrodes 18 having different polarities 162 to deliver electrical stimulation pulses to the target nerve tissue. In particular embodiments in which stimulation lead 14 includes a single stimulating electrode 18, such as a single stimulating electrode 18 at the tip of stimulation lead 14 for example, electric fields may be generated between the single stimulating electrode 18 and a terminal or other electrical contact associated with stimulation source 12. Stimulation parameters may also include a pulse shape, for example, biphasic cathode first, biphasic anode first, or any other suitable pulse shape. Stimulation parameters are not limited to the preceding but may include any suitable parameters known to those skilled in the art.

The polarity for an stimulating electrode 18 at a time 164 beginning a corresponding stimulation pulse or sub-interval within a stimulation pulse may be a relatively positive polarity 162, a relatively negative polarity 162, or an intermediate polarity 162 between the relatively positive polarity 162 and relatively negative polarity 162. For example, the relatively positive polarity 162 may involve a positive voltage, the relatively negative polarity 162 may involve a negative voltage, and the relatively intermediate polarity 162 may involve a zero voltage (i.e. “high impedance”). As another example, the relatively positive polarity 162 may involve a first negative voltage, the relatively negative polarity 162 may involve a second negative voltage more negative than the first negative voltage, and the relatively intermediate polarity 162 may involve a negative voltage between the first and second negative voltages. The availability of three distinct polarities 162 for an stimulating electrode 18 may be referred to as “tri-state” electrode operation. The polarity 162 for each stimulating electrode 18 may change for each of the sequence of times 164 corresponding to stimulation pulses or to sub-intervals within a stimulation pulse according to the stimulation parameters specified for the stimulation set 160. For example, as is illustrated in FIG. 9 for an example stimulation set 160 for a stimulation lead 14 with sixteen stimulating electrodes 18, the polarities 162 of the sixteen stimulating electrodes 18 may change for each of the sequence of times 164. In the example of FIG. 9, a relatively positive polarity 162 is represented using a “1,” a relatively intermediate polarity 162 is represented using a “0,” and a relatively negative polarity 162 is represented using a “−1,” although any values or other representations may be used.

FIG. 10 illustrates a number of example stimulation programs 166, each including a number of stimulation sets 160. One or more simulation programs 166 may be set up to provide electrical stimulation of target nerve tissue. As described above, each stimulation set 160 specifies a number of stimulation parameters for the stimulation set 160. In one embodiment, within each stimulation program 166, stimulation system 16 consecutively executes the sequence of one or more stimulation sets 160 associated with stimulation program 166. The sequence may be executed only once, repeated a specified number of times, or repeated an unspecified number of times within a specified time period. For example, as is illustrated in FIG. 11 for the third example stimulation program 166c including eight stimulation sets 160, each of the eight stimulation sets 160 is consecutively executed in sequence. Although the time intervals 168 (t₁-t₀, t₂-t₁, etc.) during which the stimulation sets 160 are executed are shown as being equal, the present invention contemplates a particular stimulation set 160 being executed over a different time interval 168 than one or more other stimulation sets 160 according to particular needs.

Although stimulation system 16 is illustrated for example as accommodating up to twenty-four stimulation programs 166 each including up to eight stimulation sets 160, the present invention contemplates any number of stimulation programs 166 each including any number of stimulation sets 160. For example, in a very simple case, a single stimulation program 166 may include a single stimulation set 160, whereas in a more complex case twenty-four stimulation programs 166 may each include eight stimulation sets 160.

In one embodiment, stimulation system 16 executes only a single stimulation program 166 in response to user selection of that stimulation program for execution. In another embodiment, during a stimulation period, stimulation system 16 executes a sequence of pre-programmed stimulation programs 166 for each stimulation lead 14 until the stimulation period ends. Depending on the length of the stimulation period and the time required to execute a sequence of stimulation programs 166, the sequence may be executed one or more times. For example, the stimulation period may be defined in terms of a predetermined number of cycles each involving a single execution of the sequence of stimulation programs 166, the sequence of stimulation programs 166 being executed until the predetermined number of cycles has been completed. As another example, the stimulation period may be defined in terms of time, the sequence of stimulation programs 166 being executed until a predetermined time interval has elapsed or the patient or another user manually ends the stimulation period. Although a sequence of stimulation programs 166 is described, a single stimulation program being executed one or more times during a stimulation period according to particular needs. Furthermore, the present invention contemplates each stimulation program 166 being executed substantially immediately after execution of a previous stimulation program 166 or after a suitable time interval has elapsed since the completion of the previous stimulation program 166.

Where stimulation system 16 includes multiple stimulation leads 14, stimulation programs 166 for one stimulation lead 14 may be executed substantially simultaneously as stimulation programs 166 for one or more other stimulation leads 14, may be alternated with stimulation programs 166 for one or more other stimulation leads 14, or may be arranged in any other suitable manner with respect to stimulation programs 166 for one or more other stimulation leads 14.

In general, each stimulation program 166 may, but need not necessarily, be set up for electrical stimulation of different nerve tissue. As an example, for electrical stimulation of the brain, one or more stimulation programs 166 may be set up for therapeutic electrical stimulation of certain nerve tissue in the brain and one or more other stimulation programs 166 may be set up for electrical stimulation certain other nerve tissue in the brain.

The present invention contemplates any suitable circuitry within stimulation source 12 for generating and transmitting signals for electrical stimulation of target nerve tissue within a person's body. Example circuitry that may be suitable for use is illustrated and described in U.S. Pat. No. 6,609,031 B1, which is hereby incorporated by reference herein as if fully illustrated and described herein.

Although the present invention has been described with several embodiments, a number of changes, substitutions, variations, alterations, and modifications may be suggested to one skilled in the art, and it is intended that the invention encompass all such changes, substitutions, variations, alterations, and modifications as fall within the spirit and scope of the appended claims.

1. An electrical stimulation lead adapted for implantation in a person's body to provide therapeutic electrical stimulation of target nerve tissue within the person's body, comprising:
one or more stimulating electrodes integrated into the stimulation lead, adapted for implantation in the person's body with the stimulation lead, and operable to provide electrical stimulation to target nerve tissue within the person's body; and
one or more sensing electrodes integrated into the stimulation lead, adapted for implantation in the person's body with the stimulation lead, and operable to detect electrical signals produced by nerve cells within the person's body to facilitate precise positioning of the one or more stimulating electrodes proximate the target nerve tissue during implantation of the stimulation lead.

2. The stimulation lead of claim 1, wherein:
the stimulation lead is adapted to be coupled to a monitoring system; and
the one or more sensing electrodes are operable to transmit signals reflecting
the detected electrical signals to the monitoring system for determination of the precise location of the one or more sensing electrodes within the person's body based at least on the signals reflecting the detected electrical signals.

3. The stimulation lead of claim 1, wherein:
each sensing electrode is a semi-microelectrode; and
each stimulating electrode is a macroelectrode.

4. The stimulation lead of claim 1, wherein:
the stimulation lead is a percutaneous lead;
the one or more stimulating electrodes comprise a plurality of circumferential electrodes spaced apart from each other along a length of a stimulating portion of the stimulation lead; and
the one or more sensing electrodes comprise a plurality of electrodes spaced apart from one another around the circumference of the stimulation lead at its tip.

5. The stimulation lead of claim 1, wherein:
the stimulation lead is a percutaneous lead;
the one or more stimulating electrodes comprise a plurality of circumferential electrodes spaced apart from each other along a length of a stimulating portion of the stimulation lead; and
the one or more sensing electrodes comprise a plurality of electrodes spaced apart from one another around the circumference of the stimulation lead between two of the plurality of stimulation electrodes.

6. The stimulation lead of claim 1, wherein:
the stimulation lead is a paddle lead;
the one or more sensing electrodes are directional electrodes located on a surface of the stimulation lead and adapted to detect electrical signals produced by nerve cells within the person's body in a direction generally perpendicular to the surface of stimulation lead; and
the one or more stimulating electrodes are directional electrodes located on the surface of the stimulation lead and adapted to deliver electrical energy to the target nerve tissue in a direction generally perpendicular to the surface of stimulation lead.

7. The stimulation lead of claim 1, wherein the electrodes are separated from each other by one or more dielectric materials.

8. The stimulation lead of claim 1, wherein the target nerve tissue comprises one of:
brain stem tissue;
spinal cord tissue; and
peripheral nerve tissue.

9. A stimulation system for providing therapeutic electrical stimulation to target nerve tissue in a person's body, comprising:
a stimulation source operable to generate and transmit electrical stimulation pulses;
an electrical stimulation lead adapted for implantation into a person's body for electrical stimulation of target nerve tissue within the person's body, the stimulation lead comprising:
one or more stimulating electrodes integrated into the stimulation lead, adapted for implantation in the person's body with the stimulation lead, and operable to deliver the electrical stimulation pulses to target nerve tissue within the person's body to provide therapeutic relief to a region of the person's body corresponding to the target nerve tissue; and
one or more sensing electrodes integrated into the stimulation lead, adapted for implantation in the person's body with the stimulation lead, and operable to detect electrical signals produced by nerve cells within the person's body to facilitate precise positioning of the one or more stimulating electrodes proximate the target nerve tissue during implantation of the stimulation lead.

10. The system of claim 9, operable to transmit signals reflecting the detected electrical signals to a monitoring system for determination of the precise location of the one or more sensing electrodes within the person's body based at least on the signals reflecting the detected electrical signals.

11. The system of claim 10, wherein the monitoring system comprises a computer system external to the person's body.

12. The system of claim 11, wherein the monitoring system is further operable to control one or more stimulation parameters of the electrical stimulation pulses generated by the stimulation source based at least on the received signals reflecting the detected electrical signals.

13. The system of claim 11, wherein the monitoring system is further operable to cause the stimulation source to begin generating electrical stimulation pulses based at least on the received signals reflecting the detected electrical signals.

14. The system of claim 9, wherein:
each sensing electrode is a semi-microelectrode; and
each stimulating electrode is a macroelectrode.

15. The system of claim 9, wherein:
the stimulation lead is a percutaneous lead;
the one or more stimulating electrodes comprise a plurality of circumferential electrodes spaced apart from each other along a length of a stimulating portion of the stimulation lead; and
the one or more sensing electrodes comprise a plurality of electrodes spaced apart from one another around the circumference of the stimulation lead at its tip.

16. The system of claim 9, wherein:
the stimulation lead is a percutaneous lead;
the one or more stimulating electrodes comprise a plurality of circumferential electrodes spaced apart from each other along a length of a stimulating portion of the stimulation lead; and
the one or more sensing electrodes comprise a plurality of electrodes spaced apart from one another around the circumference of the stimulation lead and between two of the plurality of stimulation electrodes.

17. The system of claim 9, wherein:
the stimulation lead is a paddle lead;
the one or more sensing electrodes are directional electrodes located on a surface of the stimulation lead and adapted to detect electrical signals produced by nerve tissue within the person's body in a direction generally perpendicular to the surface of stimulation lead; and
the one or more stimulating electrodes are directional electrodes located on the surface of the stimulation lead and adapted to deliver electrical energy to the target nerve tissue in a direction generally perpendicular to the surface of stimulation lead.

18. The system of claim 9, wherein the target nerve tissue comprises one of:
brain stem tissue;
spinal cord tissue; and
peripheral nerve tissue.

19. A percutaneous stimulation lead adapted for implantation in a person's body to provide therapeutic electrical stimulation of target nerve tissue within the person's body, comprising:
a plurality of circumferential stimulating electrodes integrated into the stimulation lead, spaced apart from each other along a length of a stimulating portion of the stimulation lead, separated from each other by one or more dielectric materials, adapted for implantation in the person's body with the stimulation lead, and operable to provide electrical stimulation to target nerve tissue within the person's body; and
one or more sensing electrodes integrated into the stimulation lead, spaced apart from one another around the circumference of the stimulation lead at its tip, separated from each other by one or more dielectric materials, adapted for implantation in the person's body with the stimulation lead, and operable to detect electrical signals produced by nerve cells within the person's body to facilitate precise positioning of the one or more stimulating electrodes proximate the target nerve tissue during implantation of the stimulation lead;
the stimulation lead adapted to be coupled to a monitoring system, the one or more sensing electrodes being operable to transmit signals reflecting the detected electrical signals to the monitoring system for determination of the precise location of the one or more sensing electrodes within the person's body based at least on the signals reflecting the detected electrical signals.

20. The stimulation lead of claim 19, wherein the target nerve tissue comprises one of:
brain stem tissue;
spinal cord tissue; and
peripheral nerve tissue.
